(19) United States
(12) Patent Application Publication
(10) Pub. No.: US 2014/0099595 A1
(43) Pub. Date: Apr. 10, 2014

(54) SENSOR FOR USE IN AN APPARATUS FOR DENTAL IMPLANT FIXTURE LOCATION DETERMINATION

(71) Applicant: Abraham Taub, Rishon Le Zion (IL)

(72) Inventor: Abraham Taub, Rishon Le Zion (IL)

(21) Appl. No.: 14/044,017

(22) Filed: Oct. 2, 2013

Related U.S. Application Data
(63) Continuation of application No. PCT/IL2012/000166, filed on Apr. 16, 2012.
(60) Provisional application No. 61/478,594, filed on Apr. 25, 2011.

(51) Int. Cl. A61C 19/04; A61B 5/06; A61C 8/00

(52) U.S. Cl. CPC A61C 19/04; A61C 8/0089; A61B 5/063
USPC 433/27

(57) ABSTRACT

A sensor for use in an apparatus for accurate dental implant fixture location determination including an inductive Eddy current effect based dental implant fixture location sensor, a shield operative to cancel out the effects of variable and unpredictable capacitance generated by uncontrollable factors, and a protective casing made of bio-compatible material at least partially enveloping the sensor and a handle.

### CROSS-REFERENCE TO RELATED APPLICATIONS

This application is being filed under 37 U.S.C. 111 as a continuation application (by-pass application) of International Application Number PCT/IL2012/000166, which has an international filing date of Apr. 16, 2012 and which claims priority to the following United States provisional application for patent: Ser. No. 61/478,594 filed on Apr. 25, 2011. This application claims the benefit of the priority date Apr. 25, 2011 under 37 U.S.C. 120 as a continuation of PCT/IL2012/000166, which claims priority as previously stated. The International Application Number PCT/IL2012/000166 is co-pending at the filing of this application and includes at least one common inventor. This application incorporates the above-identified International Application and United States Provisional applications by reference in their entirety.

### TECHNOLOGY FIELD

The present apparatus and method generally relate to dental restorative or corrective work. In particular, the present apparatus relates to an apparatus for identification of implant fixture location and marking the location for further dental restorative and corrective work.

### BACKGROUND

In the field of dentistry, it is often necessary to replace native teeth by prosthetic teeth, mounted on one or more dental implants to maintain an individual ability to digest food and his or her cosmetic appearance. Dental implants are increasingly used in such procedures. Dental implant is typically composed of a metallic fixture, covered with a metallic plug or cup and it is anchored within the maxillary or mandibular bone. For implant fixture insertion the gum tissue should be opened and then holes are drilled in the patient's jaw and the implant fixtures are fixed, typically screwed, into the holes. The gum tissue is then stretched and sutured over the fixtures. At a later stage the implant fixtures receive a post that bears a thread with the help of which prosthetic teeth are attached to the post and fixed over the post.

Dental practitioners usually insert the post when the implant fixture sufficiently integrates with the recipients' jaw, a process that takes a few months. In order to determine the implant fixture location the practitioner has to make a long cut in the gum unveiling the fixtures. The plug is replaced by another plug or cover enhancing the gum healing process and a new suture enabling unobstructed access to the fixtures is made. Repeat surgical interventions increase life risk to operation sensitive patients such as patients suffering from diabetes and having poor blood coagulation, or patient suffering from different paroxysmal phenomena such as hypertonia or hypotonia, patients with pacemakers, and others.

Availability of certain procedures and apparatuses facilitating precise location of implant fixtures will allow opening a very small area of gum tissue exactly above the surgery cup of each implant fixture. This would help to avoid procedures of cutting and then suturing up of gum tissue, and reduce the number of surgical interventions.

### SUMMARY

A sensor for use in an apparatus for accurate dental implant fixture location determination including an inductive eddy current effect based dental implant fixture location sensor and a handle.

In one example, the dental implant fixture location sensor is a Planar Printed Circuit Board Inductive Coil the windings of which are arranged on a number of closely spaced planes or a single plane and all of the windings participate in sensing the eddy current. The sensor may be a reusable or a disposable sensor.

In another example of the dental implant fixture location sensor—the sensor includes a pair of shields implemented as outer layers of the Printed Circuit Board and connected to the apparatus ground. In an additional example, at least one of the shields is implemented on a separate substrate. In course of assembly with the inductive coil, the substrate could form a gap with the inductive coil facilitating better thermal isolation of the sensor. The shields protect the sensor from Human Body Capacitive proximity effects influence. The shields could be transparent to electromagnetic radiation.

Some examples of the sensor could include a core. The core could have a central segment having a diameter smaller than the rest of the core. The rest of the core could have a diameter being similar in size to the Planar Printed Circuit Board.

In a further example of the dental implant fixture location sensor the sensor is partially or fully coated by a thin layer of conductive material serving as a shield. The layer is electrically connected to the circuit ground.

For detection and determination of the dental implant fixture location, the dental implant fixture location sensor is inserted into a suitable apparatus. The apparatus with the sensor is introduced into the patient's mouth and moved in a scanning motion over, or being positioned in direct contact with the gums into which one or more dental implant fixtures have been earlier inserted. Upon determination of the dental implant fixture location a marking accessory may be operated to mark the location and assist the dental practitioner in making a smaller gum incision and reducing the subject healing time.

### GLOSSARY

The term “eddy current” as used in the present disclosure means alternating electrical currents, which can be induced to flow in any metallic materials.

The term “Q-factor” or quality factor as used in the present disclosure has its conventional meaning of the quality factor of an inductor, which is the ratio of inductors inductive reactance to its resistance at a given frequency, and is a measure of its efficiency.

The term “Planar Printed Circuit Board Inductive Coil” as used in the present disclosure relates to a single-layer or multiple-layer printed circuit board (PCB) structure.

The term “uncontrollable factors” as used in the present disclosure relates to various factors such as parasitic Human (Patient) Body Capacitance, temperature inside patient mouth, saliva, occasional sensor contacts with tongue or inner side of a cheek, gum non-uniformity, variable and unpredictable capacitance variations caused by scanning movement of the probe occasionally changing the distance between the sensor and the gum, caregiver or dentist capacitance and others. Factors that generate electronic “noise” and interfere with the output generated by the sensor.

### BRIEF LIST OF FIGURES

The method and apparatus disclosed are herein presented, by way of non-limiting examples only, with reference to the accompanying drawings, wherein like numerals depict the same elements throughout the text of the specifications. The drawings are not necessarily to scale, emphasis instead being placed upon illustrating the principles of the method and the apparatus.

FIGS. 1A and 1B n examples of the present apparatus for dental implant fixture location determination;

FIG. 2 is a simplified plan view illustration of an example of a sensor for dental implant fixture location determination;

FIG. 3 is a cross-section view of the sensor for dental implant fixture location determination of FIG. 2;

FIG. 4 is an example cross-section view of the sensor for dental implant fixture location determination of FIG. 3;

FIGS. 5 is a simplified block diagram of an example apparatus and sensor for dental implant fixture location determination;

FIG. 6 is a simplified block diagram providing additional details on different factors affecting the sensor for dental implant fixture location determination operation;

FIGS. 7A and 7B are cross-section views of some examples of sensors for dental implant fixture location determination;

FIG. 8 is a simplified plan view illustration of an example of a sensor with a shield configured as a grid or thin conductive layer;

FIGS. 9A through 9C are examples of a planar Printed Circuit Board (PCB) inductive coil sensor including a core.

FIGS. 10A-10B are examples of different shield patterns.

FIG. 11 is a cross-section view illustration of another example of a sensor for dental implant fixture location determination; and

FIG. 12 is a simplified cross section view illustration of a method for marking the location of an implant fixture in accordance with the example apparatus for dental implant fixture location determination.

### DETAILED DESCRIPTION OF EXEMPLARY EMBODIMENTS

In the following detailed description, for purposes of explanation only, numerous specific details are set forth in order to provide a thorough understanding of the present apparatus and method. It will be apparent, however, that the present system and method may be practiced without these specific details. In other instances, well-known structures and devices are schematically shown in order to simplify the drawings.

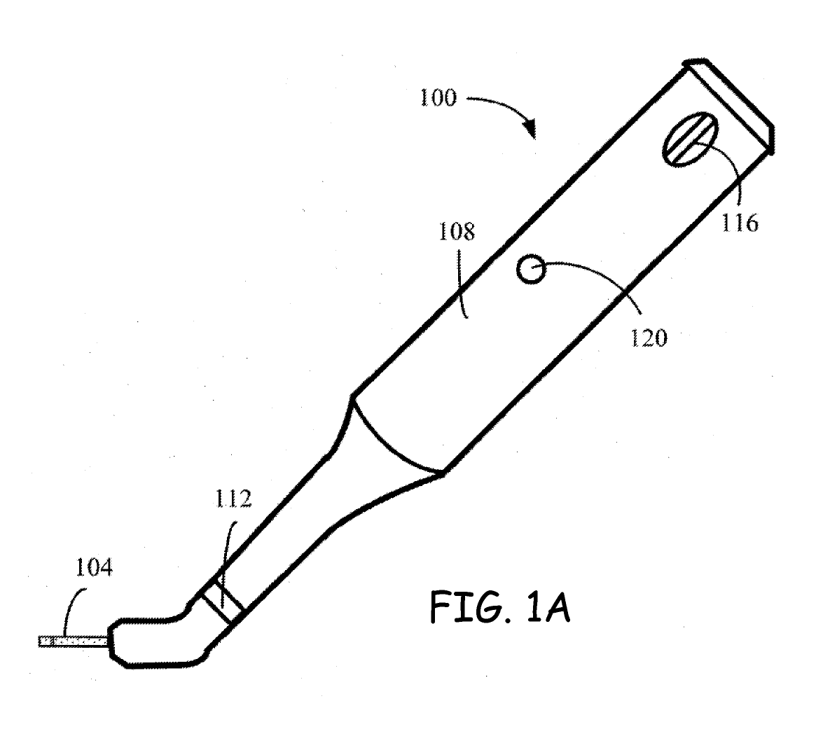

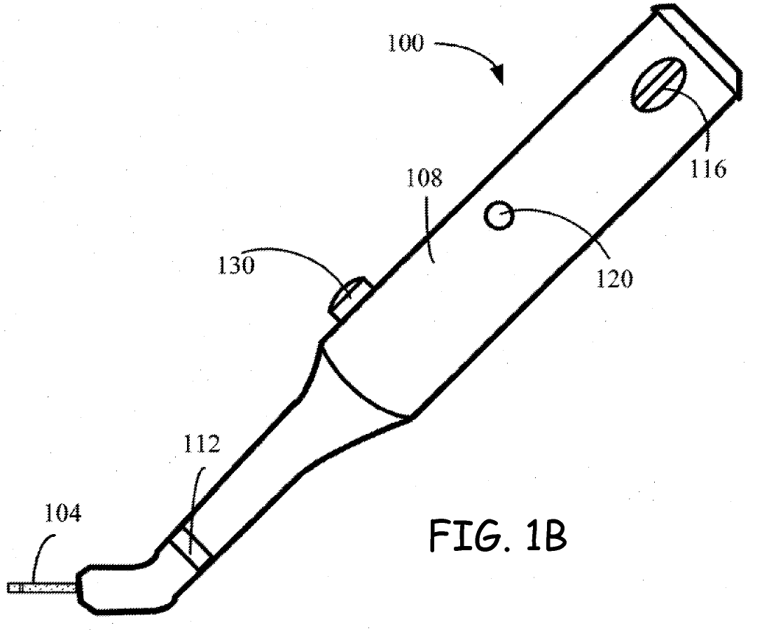

FIG. 1A is an example of the present apparatus for dental implant fixture location determination as described in the inventor's Patent Cooperation Treaty Application No. IL2010/000864, filed on Oct. 20, 2010, shown in an orientation for operating on a mandibular bone. Apparatus 100 includes an inductive dental implant fixture location sensor 104, removably inserted into a handle 108, a light signal indicator 112, an audio signal indicator 116 and could include a gum tissue marking or coagulation activation button 120. Handle 108 could be a convenient to hold, fitting in a palm of the hand case containing control and processing electronics, primary or rechargeable batteries providing power to apparatus 100. Alternatively, apparatus 100 could receive power supply from a regular network receptacle. In one example illustrated in FIG. 1A dental fixture location sensor 104 is a detachable sensor and when in use it is coupled to handle 108 by inserting it into the handle. Sensor 104 insertion or extraction provides an electrical connection to control and processing electronics and switches ON or OFF apparatus 100. Light signal indicator 112 could be a dual or more color Light Emitting Diode (LED) and the audio signal indicator 116 could be a simple buzzer.

In another example, shown in FIG. 1B, the switching function of sensor 104 could be disabled and a push button 130 could be used to switch ON or OFF apparatus 100 as well as reset apparatus operation. Switch 130 functionality could be established for example, by the time it is pushed down.

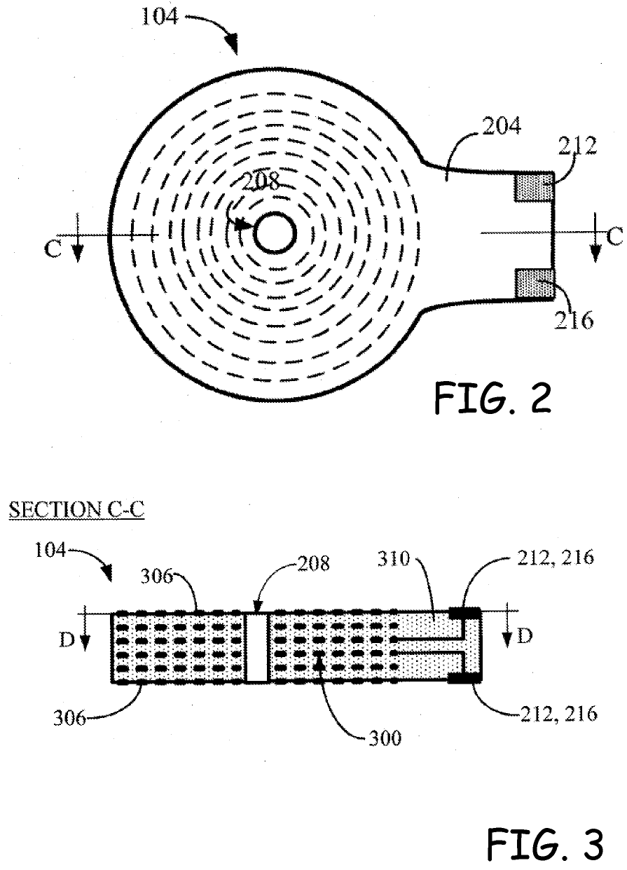

FIG. 2 is a simplified plan view illustration of an example of a sensor for dental implant fixture location determination. Generally, sensor 104 is similar to the sensor described in the applicant's Patent Cooperation Treaty Application No. IL2010/000864, filed on Oct. 20, 2010. Sensor 104 is a planar Printed Circuit Board (PCB) inductive coil encapsulated into a casing 204 made of a biocompatible material. Sensor 104 includes a centrally positioned opening 208 and a pair or more of electric contacts 212 and 216. Contacts 212 and 216 enable electrical communication with control and processing electronics located in handle 108, when sensor 104 is inserted into the handle (FIG. 1). As noticed above in some examples sensor 104 could also serve as apparatus 100 ON/OFF switch. Insertion of sensor 104 into handle 108 switches ON apparatus 100. Extraction of sensor 104 from handle 108 could switch OFF apparatus 100, eliminating the need in any dedicated ON/OFF switch. When a practitioner is performing a treatment to the same patient and sensor 104 is used as apparatus 100 ON/OFF switch, apparatus electronics could switch OFF after a preset time power supply, which could be a battery located in handle 108, extending battery life. Sensor 104 could be implemented as a disposable or reusable sensor. Reusable sensor 104 could contain non-active, non-reactive and/or temperature-insensitive materials rendering sensor 104 fully sterilizable. When sensor 104 is implemented as a disposable dental implant fixture location sensor, it could include a fuse preventing repeat use of the disposable sensor.

FIG. 3 is an example cross-section view of sensor for dental implant fixture location determination of FIG. 2. Sensor 104 is a planar Printed Circuit Board (PCB) inductive coil 300 in which windings 306 are evenly distributed between all available PCB layers on a substrate 310, although in some examples the windings could be located in a single plane. The windings are connected serially and they are forming the coil 300. Contacts 212 and 216 could be deposited on one or both sides of the sensor or on the side surfaces of the sensor.

Unlike the common three-dimensional cylindrical form coil, in which only the windings close to the tissue (mandible/maxilla) into which metallic implant is inserted, would participate in sensing the eddy currents, in the planar printed circuit board inductive coil where the coil windings located in different layers of the multilayer printed circuit board are very close to each other. Present multilayer PCP production technologies enable spacing of 10 micron to 25 micron between the different layers of windings. Because of this, all windings 306 of coil 300 could participate in sensing eddy currents induced in dental implants. The described structure substantially increases the sensitivity of the planar printed circuit board inductive coil. For all practical purposes a multilayer planar PCB inductive coil could be considered as a sensor all windings of which are in a single plane.

FIG. 4 is a cross-section view of sensor for dental implant fixture location determination of FIG. 3. Windings 306 of coil 300 could have a spiral shape, although other coil 300 windings arrangements are possible.

In one embodiment sensor 104 is a reusable sensor, made of non-active, non-reactive and/or temperature-insensitive materials and can be readily attached/detached to/from handle 108 for sensor sterilization.

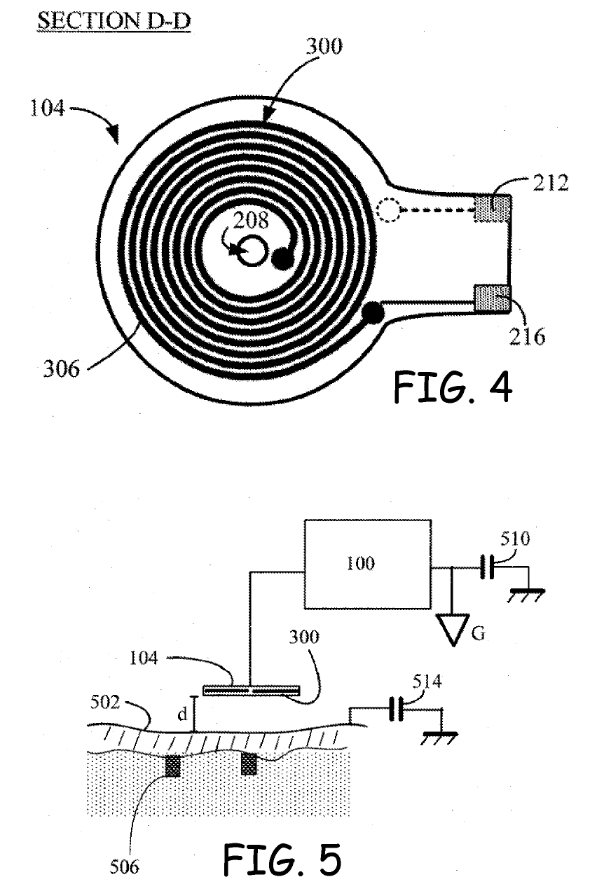

FIG. 5 that is a simplified block diagram of an example apparatus and sensor for dental implant fixture location determination in operation. Although sensor 104 could be operated by sliding it over a gum tissue surface 502 and maintaining a contact with the gum tissue surface 502, an air gap designated by the letter (d) could be occasionally formed between the surface of subject's gum tissue 502 and sensor 104 inductive coil 300. Numerals 510 and 514 indicate respectively certain parasitic capacitance existing between apparatus 100 and general ground and between the patient and general ground.

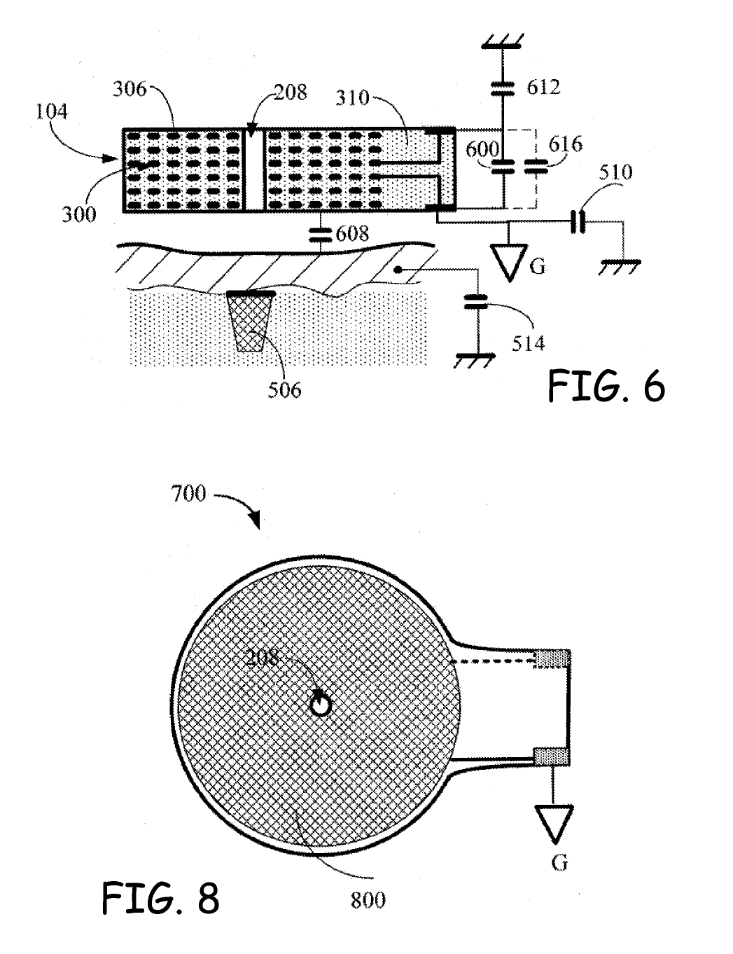

The coil is driven by an Alternating Current (AC) and generates an oscillating magnetic field that induces eddy currents in the located under the gum tissue metallic insert 506. The field induced eddy currents reduce the Q-factor of the coil, which in turn increases the frequency of the generated signal. Generation of such signal is achieved by connection of a Reference Capacitor 600 (FIG. 6) in parallel with the sensor 104 coil 300 to the same oscillation circuit. This connection creates a frequency generator. Under these conditions the sensor converts its Q-factor relative to its displacement from the insert into a change of impedance of the resonant contour. This change in impedance causes a respective change in oscillation frequency of the frequency generator. Such frequency change, being properly computed indicates the displacement (approaching to or distancing from) the insert.

FIG. 6 is a simplified block diagram providing details on other factors affecting the sensor for dental implant fixture location determination operation. The outer windings of the sensor coil could serve as a parasitic Human (Patient) Body Capacitive Proximity Sensor. Being connected in parallel with the Reference Capacitor 600, these additional parasitic capacitors 510, 514 and 612 change the measured frequency according to proximity to the gum tissue surface (gap (d) FIG. 5), tongue or inner side of a cheek. Distance (d) affects patient's body capacitance 608 and together with other parasitic capacitances such as dentist's body capacitance 618 they form together additional capacitance 616, connected in parallel with the reference capacitor 600. Other uncontrollable factors such as for example, saliva and/or the subject's body conditions, variable and unpredictable capacitances that could exist between each conductive layer 306 of sensor 104 generate electronic noise interfering with the output generated by sensor 104.

The uncontrollable factors cause variations or changes in the generated frequency, changes that could be wrongly interpreted and cause mistakes in insert location detection. Another uncontrollable factor is associated with the changes of temperature of the sensor when being inserted in the mouth of the patient. These changes are relatively slow and could be measured and taken into account for future calculation of the oscillator frequency as a function of the displacement of the sensor from the implant only.

The present apparatus utilizes changes in coil resistance to derive temperature data and compensate for temperature changes caused oscillator frequency changes. The oscillations could be periodically discontinued or switched off for a couple of millisecond and coil resistance, which depends on temperature measured. The resistance measurement loop could be included into the control and processing electronics, located in handle 108 (FIG. 1). Alternatively, temperature sensors such as thermocouples or thermistors could be incorporated into the sensor.

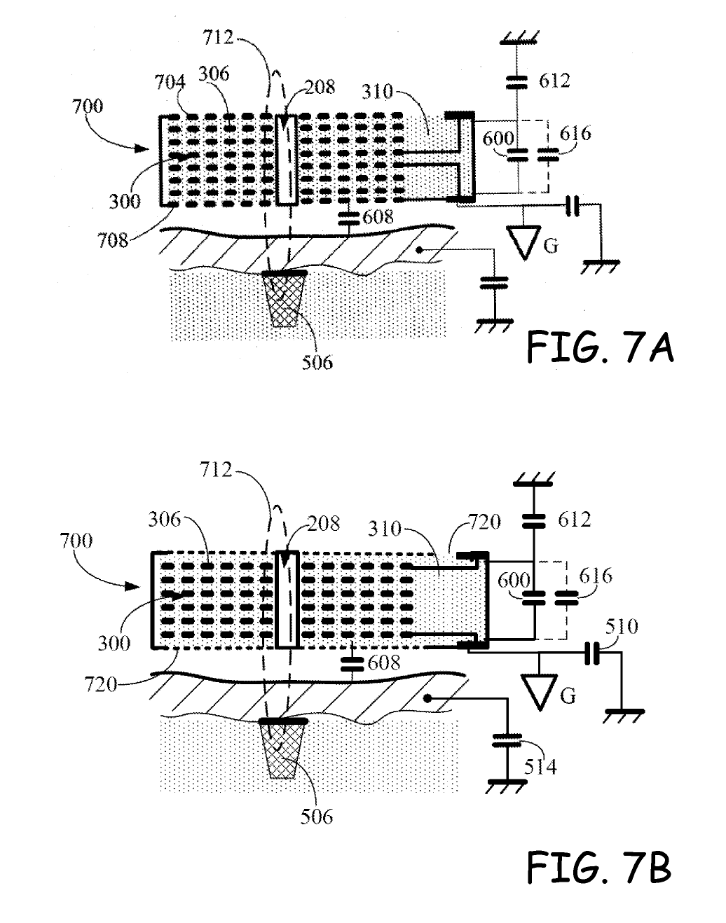

The author of the current application has conducted experimentation to cancel out the effects of variable capacitance generated by uncontrollable factors as described above. The experimentation results led to a development of a sensor containing a shield operative to cancel out the effects of variable and unpredictable parasitic capacitance generated by uncontrollable factors. FIGS. 7A and 7B are cross-section views of some examples of sensors for dental implant fixture location determination. Sensor 700 includes two shields 704 and 708 and the planar printed circuit board inductive coil 300 windings 306 are located between the two electrostatic shields 704 and 708. The two shields 704 and 708 are connected to the apparatus ground.

Shields 704 and 708 (FIG. 7A) could be implemented using the printed circuit board production technology and have a spiral shape similar to windings 306 and in some cases overlapping the coil windings and vias forming a conducting pathway between two or more winding layers as shown in FIG. 4 or as a grid or hatching 800 covering as shown in FIG. 8 the outer surface of sensor 700. Generally, as it will be shown below, other shield patterns could bring the desired results. Shields 704 and 708 could be at least partially or completely transparent to electromagnetic radiation. Properly chosen geometry of the shields conductors will not have significant influence on the electromagnetic permeability of the sensor 700 inductive loop of which is illustrated by the dashed line 712. In another example, the shield is implemented as a thin conductive coating 720 or even a layer of conductive paint.

In course of sensor 700 operation, the shields exclude different capacities (510, 514, 608, 612, and 616) (FIG. 6) responsible for the capacitive proximity effect from the capacitive coupling loop with summary capacity 616, such that only stable reference capacitor 600 (FIG. 6) participates in the sensor resonant contour. Sensor 700 structure including the described shields almost completely alleviates capacity influenced sensor reading variations caused by the uncontrollable factors. The insert location detection process becomes stable and the frequency changes affecting the accuracy of insert location determination are eliminated.

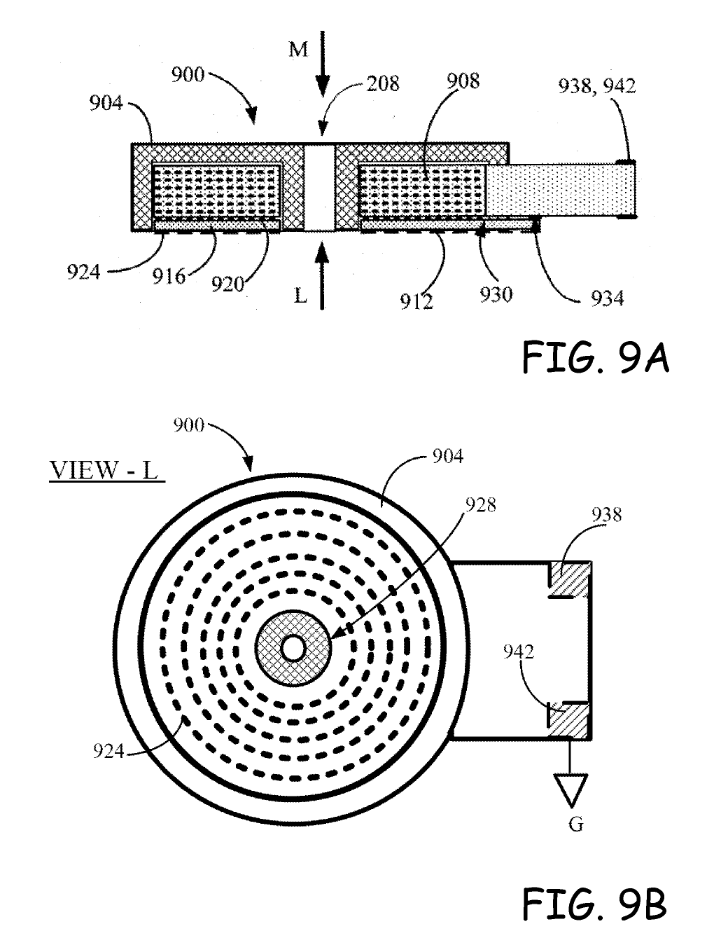

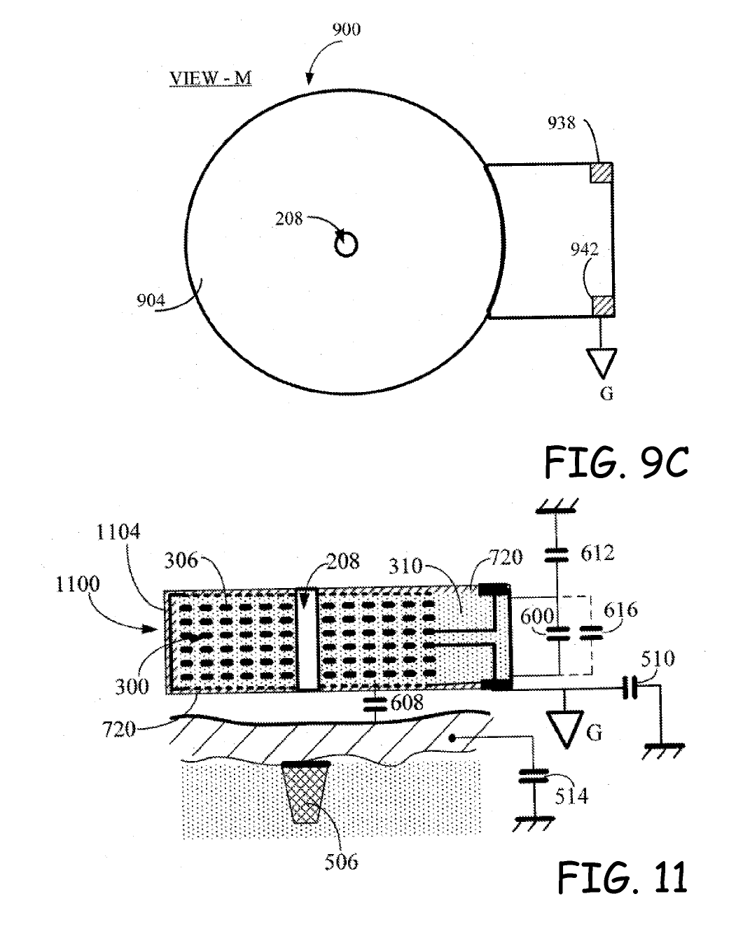

In some examples the sensors suitable for dental implant fixture location determination could include a core. FIG. 9A is a cross-section view of an example of such a sensor for dental implant fixture location determination. Sensor 900 includes a core 904, a planar Printed Circuit Board (PCB) inductive coil 908 and two shields. One of the shields 912 is implemented as an additional planar printed circuit board inductive coil located on an additional and separate substrate 916 with windings 920 facing inductive coil 908, similar or identical to the windings of coil 908, and windings 924 located on the opposite side of planar printed circuit board inductive coil 916 serving as a shield. Windings 924 could be different from windings 920 and could have a pattern different from what is understood as windings. Core 904 could have a central segment 928 having a diameter smaller than the rest of the core. The rest of the core could have a diameter being similar in size to the Planar Printed Circuit Board or large. Typically, the core would be made of a ferromagnetic material and the core segment having a diameter similar in size to the Planar Printed Circuit Board could serve as a second shield. The shields protect the sensor from Human Body Capacitive proximity effects influence. When the rest of the core has a diameter larger than the size to the Planar Printed Circuit Board it could provide additional shield to the sides of the sensor. The shields could be transparent to electromagnetic radiation.

The assembly of the additional planar printed circuit board inductive coil 916 with coil 908 could be conducted such as to form an air gap 930 with the inductive coil 908. The gap could facilitate better thermal isolation of the sensor from the subject (patient) body. Shield 924 could be soldered to a protrusion or stub 934 connected to general ground. The size of the protrusion could be used to establish the desired gap 930. Contacts 938 and 942 facilitate electrical communication with control and processing electronics located in handle 108, when sensor 900 is inserted into the handle (FIG. 1).

FIGS. 9B and 9C are examples of top and bottom views of the inductive coil sensor of FIG. 9A. Contacts 938 and 942 could be located on one or both sides of sensor 900 and are shown in FIG. 9B in broken lines.

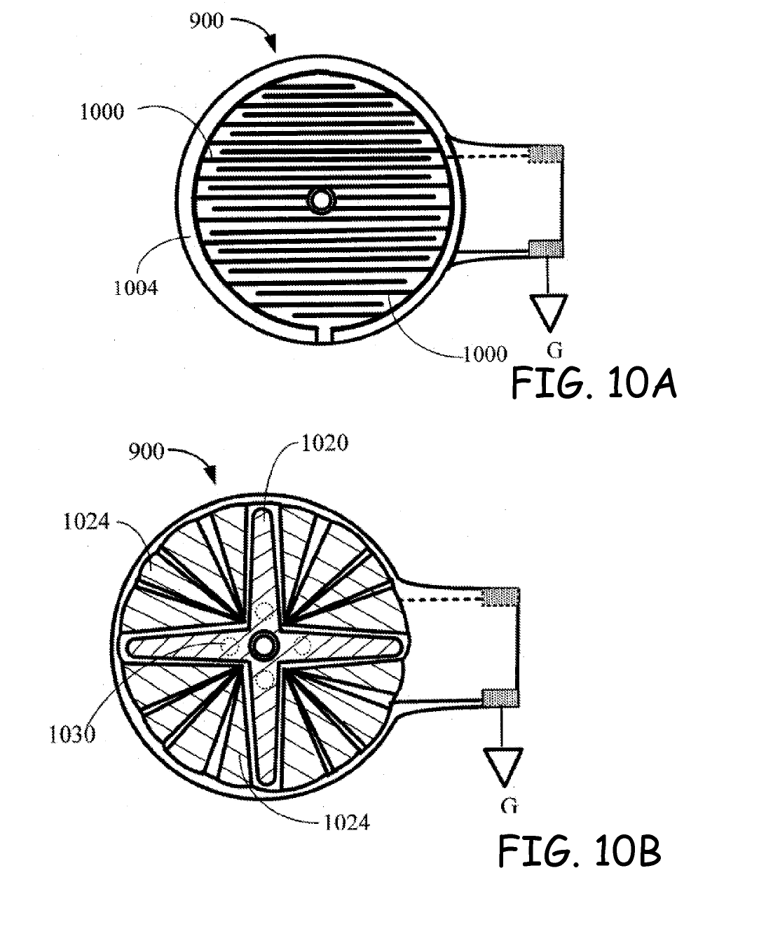

Windings 924 of the electrostatic shield could be implemented in a variety of different patterns and shapes. FIGS. 10A-10B are examples of different shield patterns. FIG. 10A is an example of a pattern of windings 924 implemented as a series of interleaving stripes 1000 connected to a common having an open ring shape conductor 1004 located at the rim of the coil. The width of stripes 1000 and the spacing between them could be selected to provide optimal shielding results.

FIG. 10B is an example of windings 924 pattern implemented in a cross like Pattern 1020 and circular segments 1024 filling in the surface area between the elements of the cross. The size and location of the cross or other similar element could be selected such as to overlap in space with coil 908 vias 1030 shown in broken lines. Shield elements location selected to overlap in space with vias 1030 of coil 908 could further reduce potential and unpredictable influence by the uncontrollable factors on electronic noise interfering with the output generated by sensor 900 or similar. It is clear that the patterns of shields disclosed in relation to sensor 900 are mutatis mutandis applicable to the earlier described sensors 104 and 700 and could be implemented without introduction of an additional and separate PCB.

The shields are typically made of conductive materials, such as metal and are exposed to contact with gums, saliva and other factors existing in human mouth. Generally, the shields and especially these of the repeat use sensors could be coated by noble metals such as gold, platinum or similar. The author of the current application has developed, as shown in FIG. 11, a protective coating 1104 for the above described sensor and other similar sensors. For example, sensor 1108 could include shields 704 and 708 in FIG. 7A or 720 or any one of shields shown in FIGS. 10A through 10B and a protective coating 1004 implemented as a casing. Casing or coating 1004 at least partially encapsulates the sensor protecting the sensor from the above described effects. The casing would typically be made of a biocompatible material the properties of which may be varied by addition of different material. The properties may include coating conductivity, dielectric constant and other properties. Such materials, for example, include but are not limited to plastic polymers (PVC, Polypropylene, Polystyrene, etc.), Silicone loaded by different materials, and similar materials.

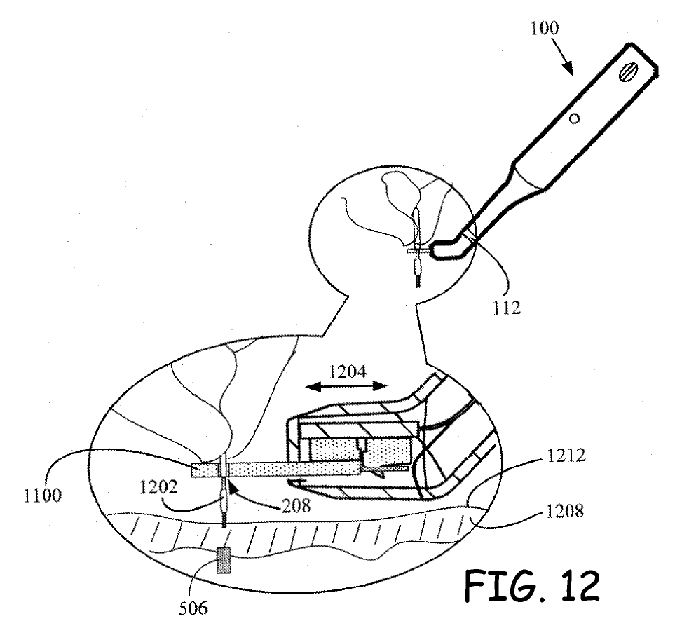

Casing 1004 could snugly adhere to at least a portion of one or more outer surfaces of the sensors to form a tight coating, although enabling access through centrally positioned opening 208 for a gum marking blade 1202 (FIG. 12) or an inkjet marking material distributor. The marking blade could be such as a thin 1 mm diameter disposable tube-like blade, similar to a known in the art biopsy punch to mark the exact location of a fixture 506 as depicted in FIG. 12.

The thickness of casing 1104 walls could be smaller than 2 mm, commonly in the range between 0.1 mm to 0.9 mm, more commonly between 0.2 mm to 0.7 mm and even more commonly between 0.1 mm and 0.4 mm. The thickness of different walls could be different and generally not sensitive to production tolerances.

Sensor 1100 could be implemented as a reusable or disposable sensor and the biocompatible material could be selected from materials that are readily sterilizable.

For detection and determination of the dental implant fixture location, as shown in FIG. 10, apparatus 100 with the sensor 1100 or any other sensor described above is introduced into a patient mouth and displaced in a scanning motion as schematically shown by arrow 1204 relative to jaw 1208 into which one or more dental implant fixtures 300 have been earlier inserted. Sensor 1100 is slid over the gum tissue typically being in contact with the gum, which covers the fixtures location and because of this, the fixtures cannot be visually detected.

Indicators, such as audio signal indicators could be employed to sound an audio signal indicating the proximity of sensor 1100 (FIG. 11) to the target fixture. Light indicator 112 and in particular the color of the light emitted by the light indicator could be used to indicate the relative proximity of the sensor 1100 with respect to the fixture 506. For example, a blinking light could be indicative of movement of sensor 1100 relative to dental implant fixture 506, whereas a continuous (non-blinking) light could be indicative of sensor 1100 being stationary relative to dental implant fixture 506.

When based on the light or audio signal the practitioner identifies the dental implant fixture 506 location he or she could hold sensor 1100 in position as determined, for example, by the color of the light emitted by light indicator 112. A marking blade 1202, such as a thin 1 mm diameter disposable tube-like blade, similar to a known in the art biopsy punch, could be inserted through opening 208 in sensor 1100 or any other described above sensor and driven through the gum surface 1212 and the gum itself over implant fixture 506 to mark the exact location of fixture 506.

The apparatus described enables fast, reliable, and simple identification of the dental implant fixture location. The practitioner has to perform a small incision in order to access a plug covering the fixture and replace it with a similar healing cup or insert the prosthesis holding abutment.

Accurate determination of fixture location minimizes the number of surgical interventions, reduces risk to operation sensitive patients such as patients suffering from diabetes and having poor blood coagulation, or patient suffering from different paroxysmal phenomena such as hypertonia or hypotonia patients with pacemakers.

A number of embodiments have been described. Nevertheless, it will be understood that various modifications may be made without departing from the spirit and scope of the method. Accordingly, other embodiments are within the scope of the following claims:

What is claimed is:

1. A sensor for an apparatus for accurate dental implant fixture location determination, said sensor comprising:
a planar printed circuit board inductive coil including a plurality of windings, the windings being arranged in a single plane located between two electrostatic shields;
the windings forming a portion of an oscillating circuit that generates a frequency, which is operative to induce an eddy current in a metal fixture when proximate to said metal fixture; and
wherein each of the two electrostatic shields and one end of the planar printed circuit board inductive coil is connected to apparatus ground.

2. The sensor according to claim 1, wherein each of the two shields is operative to cancel out effects of variable and unpredictable capacitive coupling loop generated by uncontrollable factors.

3. The sensor according to claim 1, further comprising a core made of a ferromagnetic material, the core including a segment with a diameter similar in size to the planar printed circuit board and the core serves as a second shield.

4. The sensor according to claim 1, wherein a layer of isolating material separates each of the two shields and the inductive coil windings.

5. The sensor according to claim 1, wherein the shields are at least partially electromagnetically transparent.

6. The sensor according to claim 1, wherein the sensor is encapsulated into a casing made of a biocompatible material.

7. The sensor according to any one of preceding claim 1, wherein the sensor also includes a centrally positioned opening facilitating a gum marking blade introduction.

8. The sensor according to claim 1, wherein the sensor is at least one of a group consisting of reusable or disposable sensors.

9. The sensor according to claim 1, wherein the sensor is readily sterilizable.

10. The sensor according to claim 1, wherein changes in the inductive coil resistance are used to derive temperature data and compensate for temperature changes causing oscillator frequency changes.

11. The sensor according to claim 1, further comprising at least one of a group of temperature sensors incorporated into the sensor consisting of a thermocouple or thermistor.

12. An apparatus for accurate dental implant fixture location determination, said apparatus comprising:
a sensor operative to inductively induce eddy currents in a dental implant when the sensor is brought proximate to the dental implant location;
a handle; and
wherein the sensor is a planar printed circuit board inductive coil with windings arranged on at least one single plane and at least one shield operative to cancel out effects of variable and unpredictable capacitance generated by uncontrollable factors; and
wherein when the sensor is coupled to the apparatus, operates to turn the apparatus on.

13. The apparatus according to claim 12, wherein the sensor is at least one of a group of sensors consisting of reusable or disposable sensors.

14. The apparatus according to claim 12, wherein the sensor is readily sterilizable.

15. The apparatus according to claim 12, wherein the sensor further comprises an opening facilitating implant fixture location marking accessory introduction.

16. The apparatus according to claim 12, wherein said handle also comprises a varying color LED light indicator and an audio indicator, said indicators operative to indicate movement and relative proximity of the dental implant fixture location sensor to the fixture.

17. The apparatus according to claim 12, further comprising control and processing electronics located in the handle.

18. The apparatus according to claim 12, further comprising a temperature compensating loop operative to measure the planar printed circuit board inductive coil resistance and communicate the resistance to control and processing electronics.
